(19) United States
(12) Patent Application Publication
(10) Pub. No.: US 2018/0292298 A1
(43) Pub. Date: Oct. 11, 2018

(54) GAMMA STERILIZED DEXTRAN SOLUTIONS AND METHODS OF USE

(71) Applicant: General Electric Company, Schenectady, NY (US)

(72) Inventors: Reginald Donovan SMITH, Schenectady, NY (US); Patrick Joseph McCloskey, Watervliet, NY (US)

(21) Appl. No.: 16/010,183

(22) Filed: Jun. 15, 2018

Related U.S. Application Data
(62) Division of application No. 15/149,383, filed on May 9, 2016.
Application No. 15/149,383 is a continuation-in-part of application No. 14/254,152, filed on Apr. 16, 2014, now abandoned.
Application No. 14/254,152 is a continuation-in-part of application No. 14/565,142, filed on Dec. 9, 2014, now Pat. No. 9,709,549.
Application No. 14/565,142 is a division of application No. 12/325,672, filed on Dec. 1, 2008, now Pat. No. 8,961,787.

(51) Int. Cl. G01N 1/34; C12N 5/00; G01N 33/49

(52) U.S. Cl. CPC G01N 1/34; C12N 2500/50; G01N 33/491; C12N 5/0087

(57) ABSTRACT

Provided herein are kit for providing a gamma sterilized aqueous dextran solution that increase the efficiency of blood separation by allowing the dextran solution to be sterilized by exposure to gamma radiation while maintaining sufficient molecular weight to act as a red blood cell aggregate. Also provided are methods of use.

### CROSS-REFERENCE AND RELATED APPLICATIONS

This application is a divisional of U.S. patent application Ser. No. 15/149,383 entitled “Gamma Sterilized Dextran Solutions and Methods of Use” filed May 9, 2016, now copending, which is a continuation-in-part of U.S. patent application Ser. No. 14/254,152 entitled “Gamma Stabilized Dextran Solutions and Methods of Use” filed Apr. 16, 2014, now abandoned, and of U.S. patent application Ser. No. 14/565,142 entitled “SYSTEMS AND METHODS FOR PROCESSING COMPLEX BIOLOGICAL MATERIALS”, filed Dec. 9, 2014, now issued as U.S. Pat. No. 9,709,549 issued Jul. 18, 2017, which is a divisional application of U.S. Pat. No. 8,961,787 issued Feb. 24, 2015. The entire disclosure of U.S. application Ser. Nos. 14/254,152 and U.S. application Ser. No. 14/565,142 are incorporated herein by reference.

### BACKGROUND

Separation of red blood cells (RBC) from whole blood is commonly required prior to analysis or therapeutic use of less abundant cells, such as white blood cells or stem cells. Many conventional blood cell isolation procedures require preliminary red blood cell depletion and additional sample volume reduction by plasma removal. These steps are commonly performed in long-term cell banking and regenerative medicinal applications, where a maximal yield of nucleated blood cells is desired in a reduced volume for direct transplantation, storage for future use or further processing to enrich/purify specific cell types.

Often these methods utilize dextran as an aggregant to enhance the sedimentation of red blood cells from whole blood or similar materials. A critical part of the manufacturing process is the sterilization of the kit which is accomplished by exposure to gamma irradiation at a dose between 20 and 50 kGy, sufficient to ensure sterilization. Unfortunately, dextran in water is unstable to gamma irradiation resulting in severe molecular weight decomposition of the dextran. For example, 3,300 kD Mw dextran will decompose to less than 20 kD on exposure to only a 20 kGy dose of gamma irradiation. Furthermore, RBC sedimentation enhancement performance of the added dextran is a function of its molecular weight and is ineffective below 200 kD molecular weight. As a result the dextran solution must be sterilized separately by autoclaving or filtering the solution then reassembling with the rest of the (gamma sterilized) kit adding cost and potential contamination during manufacturing or customer use.

As such there is a need for gamma sterilized dextran solutions which will allow incorporation of the dextran more directly into the handling and manufacturing process to insure stability but also reduce risk from subsequent handling errors and cost.

### BRIEF DESCRIPTION

In general, the methods and kits of the invention provide gamma stable dextran solutions, which can be sterilized through gamma irradiation while maintaining a sufficient molecular weight distribution to subsequently act as an effective red blood cell (RBC) aggregant. This increases the efficiency of blood separation/fractionation process as the dextran solution may be incorporated directly and seamlessly into the handling and manufacturing process.

In another embodiment, a kit is provided for producing a gamma sterilized aqueous dextran solution comprising a mixing vessel for red blood cell aggregation, dextran having an initial average molecular weight greater than 500 kD, and 2.0 to 20.0 wt % ascorbic acid or its mineral salt to the dextran.

In another embodiment a method provides for adding the gamma sterilized aqueous solution to a blood sample (peripheral blood, cord blood), resulting in increased red blood cells (RBC) aggregation and sedimentation while recovering a large percentage of the total nucleated cells (TNC). The method comprises the steps of subjecting a dextran solution comprising ascorbic acid or a mineral salt of ascorbic acid to gamma radiation, adding to the blood sample, incubating to aggregate and partition RBCs, and recover TNCs.

### FIGURES

These and other features, aspects, and advantages of the present invention will become better understood when the following detailed description is read with reference to the accompanying figure.

FIG. 1 is flow diagram of the process to combine a gamma sterilized dextran solution and a sample.

FIG. 2is is a graphical representation of a device for aggregation of red blood cells using a gamma sterilized dextran solution.

FIG. 3A is a graphical representation of a process for gamma sterilization of an assembled kit comprising a mixing vessel and a receptacle containing a dextran solution.

FIG. 3B is a graphical representation of a process for gamma sterilization of a disposable kit without the receptacle assembly.

### DETAILED DESCRIPTION

The following detailed description is exemplary and not intended to limit the invention of the application and uses of the invention. Furthermore, there is no intention to be limited by any theory presented in the preceding background of the invention on the following detailed description. To more clearly and concisely describe and point out the subject matter of the claimed invention, the following definitions are provided for specific terms that are used in the following description and the claims appended hereto.

Unless otherwise indicated, the article “a” refers to one or more than one of the word modified by the article “a.” Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

“Dextran” refers to polysaccharides with molecular weights≥1000 Dalton (Da), which have a linear backbone of a-linked D-glucopyranosyl repeating units and typically have a molecular weight ranging from 3,000 Da to 2,000,000 Da. It is often classified according to molecular weight. For example dextran 500 refers to an average molecular mass of 500 kDa. Dextran 1230 refers to an average molecular mass of 1230 kDa.

“Kit” is referred to herein as one or more reactants or additives necessary for a given assay, test, or process. The kit may also include a set of directions to use the reactants or additives present in the kit, any buffers necessary to maintain processing conditions or other optional materials for using. In certain cases the kit may contain premeasured amounts of the reactants or additives for a given assay, test, or process. The kit may also contain other materials to optimize use with a device for separation of the blood cells. The kit may comprise disposable components such as molded polymeric compartments, integrated tubing and valves, which enable its intended operation.

In certain embodiments a gamma sterilized dextran solution is provided, the solution comprising of an aqueous solution of dextran, ascorbic acid or a mineral salt of ascorbic acid. In certain embodiments, the dextran solution is approximately 1 to 10 wt % of dextran in an aqueous solution. Preferably the dextran is 1 to 5wt % and more preferable 2 to 4wt % dextran in an aqueous solution. In certain embodiments the red blood cell sedimentation enhancement performance of added dextran is a function of molecular weight and it is ineffective below 200 kD molecular weight. As such in certain embodiments, after gamma irradiation the dextran has a molecular weight greater than approximately 200 kD. In preferred embodiments, the molecular weight is in a range of approximately 200-800 kD and most preferred the dextran has molecular weight in a range of approximately 400-600 kD after gamma irradiation. In certain embodiments the dose of gamma radiation is between 20 and 50 kGy, and more preferably at a dose between 25 and 45 kGy.

In certain embodiments, the ascorbic acid is a mineral salt including, but not limited to sodium ascorbate, calcium ascorbate, potassium ascorbate, magnesium ascorbate, zinc ascorbate or a combination thereof. In certain embodiments the mineral salt is sodium ascorbate. The mineral salt provides a certain level of stability for the dextran, to prevent MW degradation to level where the dextran is not an efficient RBC aggregation enhancer. As such, the ascorbic acid, or its salt, is used to provide controlled, limited MW dextran degradation.

In certain embodiments the ascorbic acid or its mineral salt added from approximately 2 to 20 wt % to an aqueous dextran in preferred embodiments from 4 to 15 wt %, and more preferable from 4 to 10 wt %. In certain other embodiments the mineral salt is added directly to dextran at approximately 2 to 20 wt %, in preferred embodiments from 4 to 15 wt %, and more preferable from 4 to 10 wt %. The ascorbic acid or its mineral salt may act as a radiation stabilizer, conserving the dextran molecular weight at a level sufficient to act as an aggregant to enhance the sedimentation of RBC.

In certain embodiments, the gamma sterilized dextran solution may further comprise buffers. The buffer comprises organic or inorganic salts that maintain a pH of 4.0 to 8.0 such as such as phosphate buffered saline (PBS). The solution may also comprise other non-toxic enhancers such as, sodium citrate, sodium succinate and combinations thereof. The use of non-toxic enhancers, the methods of aggregating blood cells and its use in connection with the system and methods are further described in aforementioned U.S. patent application, Ser. No. 14/565,142.

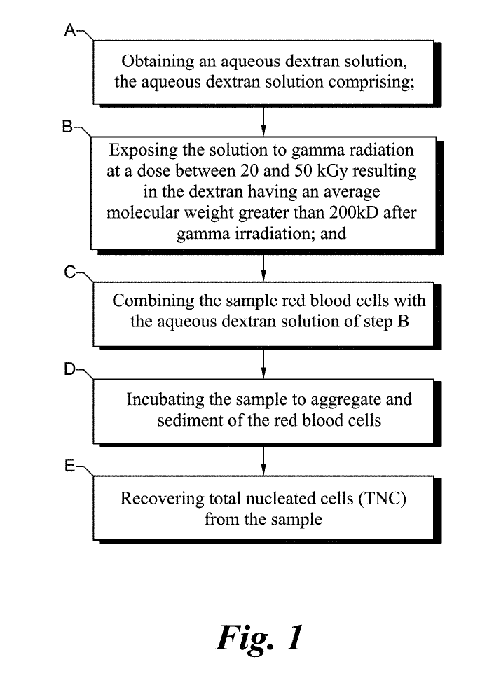

As shown in FIG. 1, in certain embodiments a method to aggregate cells in a sample comprising red blood cells (RBC) is provided comprising the steps of obtaining an aqueous dextran solution comprising 1 to 10 wt/v % of dextran where the dextran has an initial average molecular weight greater than 500 kD and 2.0 to 20.0 wt % ascorbic acid or its mineral salt to the dextran (Step A) followed by exposing the solution to gamma radiation at a dose between 20 and 50 kGy and where the dextran has an average molecular weight greater than 200 kD after gamma irradiation (Step B). After gamma irradiation, the sample comprising red blood cells and the aqueous dextran solution are combined together (Step C).

As shown further in FIG. 1, in certain embodiments the method is used to sediment cells improve the resulting recovery of an increased percentage of total nucleated cells (TNCs) from a sample comprising red blood cells (RBC). As such, after adding the RBC sample to the aforementioned gamma sterilized dextran solution in certain embodiments, the method further comprises incubation of the sample to aggregate and sediment the plurality of RBCs (Step D) and/or eventual recovering the TNC (Step E). The later steps are commonly performed in long-term cell banking and regenerative medicinal applications, where a maximal yield of nucleated blood cells is desired in a reduced volume for direct transplantation or storage for future use. In certain embodiments, the method of recovering the TNC may comprise concentration of a liquid phase prior to collection. The liquid phase comprises plasma and dextran and thus concentration may be accomplished through a number of methods including centrifugation, membrane filtration, or a combination of methods.

In certain embodiments the sample comprising the RBC is whole blood, in certain other embodiments the sample comprising the RBC is a blood component, such as but not limited to isolated blood fraction including bone marrow and mobilized peripheral blood.

In certain embodiments, a kit is provided comprising the solutes necessary for producing a gamma sterilized dextran solution. In certain embodiments, the kit comprises solutes, where the solutes are dextran, ascorbic acid or a mineral salt of ascorbic acid. The ascorbic acid or its mineral salt is presence in 2.0 to 20.0 wt % to the dextran. In certain embodiments, the dextran has a molecular weight (MW) that is sufficient to maintain a MW greater than approximately 200 kD after the solutes are incorporation into an aqueous solution and after exposure to gamma radiation. In certain embodiments, the initial molecular weight of dextran is greater than 500 kD, in preferred embodiments greater than 750 kD and most preferred greater than 1000 kD. In certain other embodiments, the initial molecular weight of dextran is between 1000 kD and 2000 kD, in preferred embodiments the initial molecular weight of dextran is approximately 1000-1500 kD.

In certain embodiments, the components of the kit are dry mixed in an amount that, when added to an aqueous solution, yields a gamma serializable dextran solution of approximately 1 to 10 wt % of dextran in an aqueous solution. Preferably the dextran is 1 to 5 wt % and more preferable 2 to 4wt % dextran when used in an aqueous solution.

In certain embodiments, the kit may further comprise buffers such as phosphate buffered saline (PBS), saline and or other non-toxic enhancers such as, sodium citrate, sodium succinate and combinations thereof. The additives may be combined in such a way that when added together in an aqueous solution, a gamma sterilized dextran solution is obtained that is still capable enhancing RBC aggregation. In certain embodiments, the kit is prepared in such a way that the individual components are provided separately. In other embodiments, the kit is prepared such that components in solid form may be premixed and supplied together. In still other embodiments, the kit is prepared such that certain components are provided in solution. In certain embodiments, the kit may further comprise components for use with a device for separation of the blood cells. In certain embodiments, the kit may comprise disposable components such as molded polymeric compartments, integrated tubing and valves, which enable its intended operation.

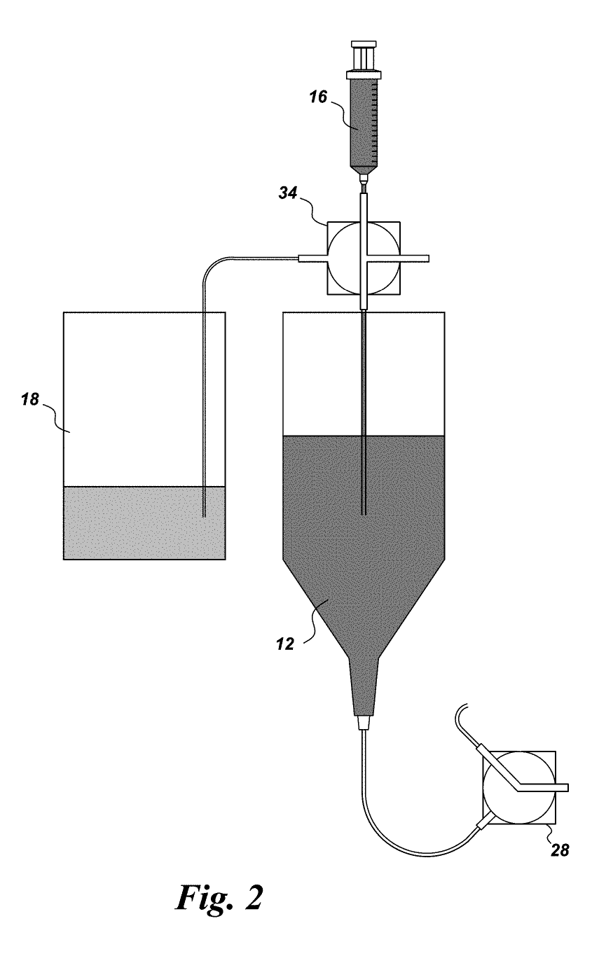

For example, as shown in FIG. 2, a disposable mixing vessel (12) may be provided that has two or more valve port opening , such as the valve (34) shown at the top of the vessel, through which a flow device, for example a syringe (16) may be used to introduce and/or withdraw materials and submaterials from vessel at various times during the blood separation process; including the RBC sample and the aqueous dextran solution. The vessel further comprises a second valve port opening (28) at the bottom of the device configured to draw off or otherwise extract sedimentary layers. Other examples of such a disposable separation device, including the vessel, are shown in the aforementioned U.S. patent application, Ser. No. 14/565142.

In certain embodiments the mixing vessel may be adapted to separate the material into aggregated submaterials, which in this example, includes aggregated RBC. The RBC are separated into a sedimentary layer, after being mixed with the aqueous dextran solution, and the flow device is adapted to draw off or otherwise extract the RBC. In certain embodiments, the mixing vessel is configured to allow a range of sample volumes between 50 to 500 ml. In the embodiment shown in FIG. 2, the mixing vessel 12 may further comprises a pick up line with a distal end located towards the bottom of vessel 12 to draw off a lowermost layer within the vessel once submaterials have separated into their respective sedimentary layers. This may be withdrawn for example through a second valve (28) The flow device may alternatively, or additionally, draw off an uppermost layer within the vessel, or one or more layers in between the lowermost and uppermost, depending on the configuration of the device 16 relative to 12.

As shown further in FIG. 2, in certain embodiments the aforementioned solutes may be provided along with the mixing vessel, flow device, and valves as part of a kit, used for RBC aggregation. The mixing vessel, flow device and valves may be disposable components which are used with the solutes, which may be provided, premeasured in dry mixed amounts, to be added to the vessel in aqueous form or, in an alternative embodiment, in a premeasured aqueous form which can be provided in or added to the receptacle (18). As such, in certain embodiments, the aqueous dextran solution may be added from the receptacle. In certain embodiments, the receptacle may be the same relative size as the mixing vessel as the aqueous dextran solution is the main volume fraction. The receptacle is in fluid communication with the mixing vessel through a valve port (34), as shown in FIG. 2, or through a separate opening.

In certain embodiments, the solutes are provided in dry form, in the kit and prior to use dissolved to for the desired aqueous solution. In certain other embodiments, the components are dissolved to form the desired aqueous solution which is added to the vessel for inclusion in the kit. The materials, as provided for in the kit, are such that it allows for sterility of the system to be maintained throughout the process.

As such, in preferred embodiments, the methods and kits of the invention to sediment blood cells generally comprise adding the gamma sterilized dextran solution to accelerate RBC sedimentation. For example, in one embodiment, a sample that includes red blood cells is treated by adding an irradiated gamma sterilized dextran solution, followed by incubation of the sample, and eventual recovery of the total nucleated cells (TNCs).

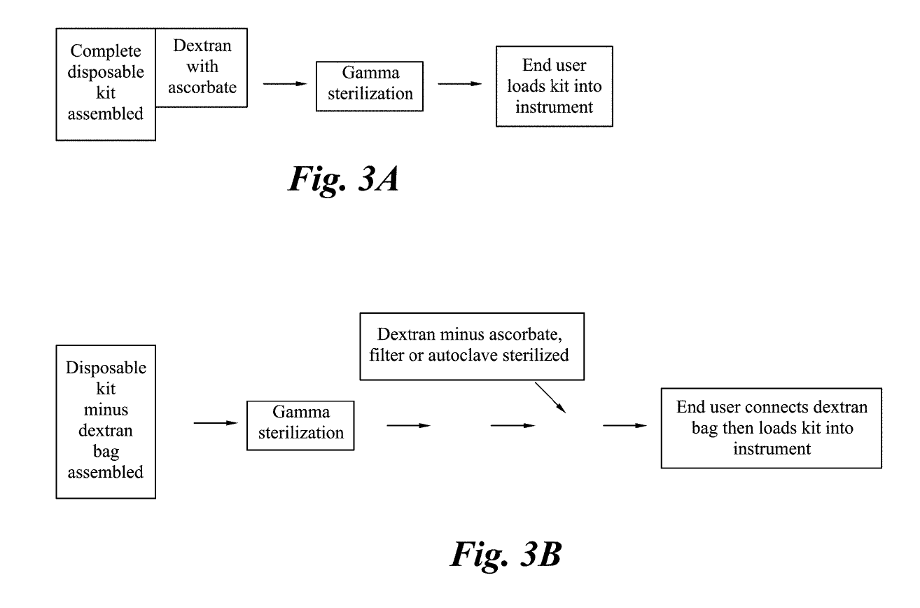

The advantage of the kit, shown in FIG. 2 is the kit may be used with the need for or the requirement that the dextran solution undergo separate sterilization; by autoclaving or filtering the solution then reassembling with the rest of the (gamma sterilized) kit. Separate sterilization may result in both additional cost and potential contamination during manufacturing or customer use. Here the dextran solution, once placed in the receptacle (18) may undergo gamma sterilization with the rest of the kit components. This is shown more clearly in FIG. 3A, where an assembled kit, containing an aqueous dextran solution, undergoes a single gamma sterilization compared to FIG. 3B which shows a two step sterilization process for the mixing receptacle and the aqueous dextran solution.

As such the gamma sterilized dextran solutions allows incorporation of the dextran more directly into the handling and manufacturing process to insure stability but also reduce risk from subsequent handling errors and cost.

In certain embodiments the RBC is added to the dextran solution under sterile processing conditions. In certain other embodiments, the dextran is added to the RBC under sterile processing conditions.

One or more of the methods of recovering a percentage of TNCs from a sample comprising red blood cells comprises adding a gamma sterilized dextran solution which has been irradiated, at a predetermined concentration, incubation of the sample, and eventually recovering the total nucleated cells.

### EXAMPLES

Practice of the invention will be more fully understood from the following examples, which are presented herein for illustration only and should not be construed as limiting the invention in any way.

Materials: Human cord blood was used for the experiments. The dextran 1230 used in this example was obtained from GE Healthcare, (Uppsala, Sweden). Ammonium formate and sodium ascorbate are available from Sigma-Aldrich (St. Louis, Mo.). Sodium citrate was obtained from Thermo Fisher Scientific (Waltham, Mass.). Pure dextran samples were prepared by dissolving approximately 37.5 mM of sodium citrate and 2.25% (w/v) of dextran in 40% (v/v) of saline (Baxter, Deerfield, Ill.) and 31.6% (v/v) of water for injection (Baxter, Ill.). Dextran and sodium citrate were allowed to dissolve for 2 hours after which the remaining saline was added to make up the desired volume. All samples were allowed to dissolve for at least 2 hr prior to analysis.

Analysis of Dextrans was Accomplished using Gel Permeation Chromatography in Combination with Multi-Angle Laser Light Scattering.

Static Light Scattering analysis in combination with aqueous phase gel permeation chromatography was achieved using an Agilent 1100 series HPLC in combination with a Wyatt Technologies Dawn EOS Multi-Angle Light Scattering Detector in-line to a Wyatt Optilab DSP Interferometric Refractive index detector. The light scattering detector collects scattered light at 18 angles upstream of the refractive index (DRI) data acquisition. The function of the DRI detector is to provide a concentration term from the RI response of a given analyte with a known refractive index increment value (DN/DC) for use in the first principle calculation equation for molar mass. The chromatography was achieved using an isocratic elution of 2 mM ammonium formate (pH 4-5). The molar mass values were obtained using the first principle calculation as derived from the Zimm formalism.

The GPC separation was achieved using 2 Tosoh PW Columns: 1-G6000 and 1-G3000 aqueous SEC columns (7.5×300 mm) in series at a flow rate of 1 ml min-1 run at ambient temperature as outlined in Table 1.

TABLE 1
Method Parameters for Gel Permeation Chromatography in combination
with Multi-Angle Laser Light Scattering/Differential Refractive
Index Detection of Dextrans.

| | |
|---|---|
| Instrument | Agilent 1100 Series HPLC/Wyatt EOS Multi-Angle |
| | Laser Light Scattering Detector w/Quasi-Elastic |
| | LS Accessory (QELS)/Opti/ab Differential |
| | Refractive Index Detector |
| Column | 1-Tosoh G3000 PW (7 × 300 mm) and 1-Tosoh |
| | G3000 PW (7 × 300 mm) |
| Mobile Phase | 2 mM Ammonium Formate pH = 4-5 |
| Flow | 1 ml/min |
| Temperature | Ambient (28-30 C.) |
| Injection | 20 ul |
| Volume | |
| DRI | 35 C. |
| Temperature | |
| DNDC | 0.145 ml/g |
| Gradient Profile | Isocratic Elution |

Table 2 shows the effects of ascorbic acid or its sodium salt added from 1.0 to 10.0 wt % to an aqueous dextran solution followed by exposure to 40 kGy dose of Gamma irradiation (Cobalt 60)

TABLE 2
Dextran solutions exposed to 40 kGy dose of gamma radiation

| | | | Gamma | |
|---|---|---|---|---|
| Sample | | % | dose | Mw after |
| # | Dextran Mw (kD) | Ascorbate | (kGy) | irradiation |
| 6 | Dextran AB (1,230) | 0 | none | 1,230 |
| 12 | Dextran AB (1,230) | 0 | 40 | 35 |
| 13 | Dextran AB (1,230) | 1 | 40 | 51 |
| 14 | Dextran AB (1,230) | 2 | 40 | 95 |
| 13 | Dextran AB (1,230) | 4 | 40 | 342 |
| 14 | Dextran AB (1,230) | 5 | 40 | 413 |
| 15 | Dextran AB (1,230) | 6 | 40 | 452 |
| 16 | Dextran AB (1,230) | 10 | 40 | 614 |

As can be seen from data in Table 3, severe Mw drop was noted even at the low 20 kGy dose of gamma radiation. However, a significant response to added wt % ascorbate versus molecular weight retention was observed notably around 4 wt %.

TABLE 3
20 kGy Experimental Results

| | | | Gamma | |
|---|---|---|---|---|
| | | Wt % | dose | Mw after |
| Sample | Dextran source | ascorbate | (kGy) | irradiation |
| control | Dextran AB (1,230) | 0 | 20 | 21 |
| 10 | Dextran AB (1,230) | 5 | 20 | 586 |
| 11 | Dextran AB (1,230) | 6 | 20 | 616 |

At the lower dose of 20 kGy (Table 3) significantly less ascorbate was required to retain molecular weight.

Dextran having a Mw of greater than 200 kD is desirable for optimal sedimentation performance. Using combination of starting high molecular weight dextran in the presence of ascorbate achieves that goal. However due to the presence of ascorbate it was still desirable to confirm the cord blood sedimentation performance.

The extent of red blood cell aggregation was measured in vitro by mixing 5 mls cord blood (sourced from New York Blood Center) in a 20 ml tube (from Globe Scientific) with 10 mls of aqueous dextran solutions from Table 4. A control sample was also prepared without the sodium ascorbate. The tube was capped and the contents mixed well by inverting the tube up and down. The contents were allowed to settle under gravity and the height of the RBC pellet was measured at 0, 10, 20, 40, and 60 minutes.

As shown in Table 4 a number of the above samples were added to cord blood to assess the RBC sedimentation performance.

TABLE 4
Sedimentation Performance

| | | | Gamma | | Aggregation (cm) | | |
|---|---|---|---|---|---|---|---|
| Sample | | % | Dose | MW after | 0 | 20 | 60 |
| # | Material | Ascorbate | (kGy) | irradiation | min | min | min |
| 1 | Dextran | 0 | none | 1,230 | 12.2 | 3.0 | 2.4 |
| 2 | Dextran | 4 | none | 1,230 | 11.9 | 2.6 | 2.0 |
| 3 | Dextran | 0 | 40 | 35 | 12.0 | 11.8 | 11.5 |
| 4 | Dextran | 4 | 40 | 342 | 12.2 | 3.2 | 2.1 |
| 5 | Dextran | 5 | 40 | 413 | 12.0 | 3.5 | 2.0 |
| 6 | Dextran | 6 | 40 | 452 | 12.0 | 4.4 | 2.1 |

Table 4 shows that gamma irradiated dextran's in the presence of sodium ascorbate aggregates RBCs to similar extents as non-irradiated dextran's with and without sodium ascorbate.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method to aggregate cells in a sample comprising red blood cells (RBC), comprising the steps of:
a. obtaining an aqueous dextran solution, the aqueous dextran solution comprising;
1 to 10 wt/v % of dextran where the dextran has an initial average molecular weight greater than 500 kD prior to gamma irradiation; and
2.0 to 20.0 wt % ascorbic acid or its mineral salt to the dextran;
b. exposing the solution to gamma radiation at a dose between 20 and 50 kGy resulting in the dextran having an average molecular weight greater than 200 kD after gamma irradiation; and
c. combining the sample comprising red blood cells with the aqueous dextran solution of step b.

2. The method of claim 1 where the dextran has an initial molecular weight greater than 750 kD.

3. The method of claim 2 where the dextran has an initial molecular weight between approximately 1000 to 1500 kD.

4. The method of claim 1 where the mineral salt is sodium ascorbate.

5. The method of claim 1 where the aqueous solution further comprises a buffer, a non-toxic enhancer or a combination thereof.

6. The method of claim 5 where the non-toxic enhancer is sodium citrate, sodium succinate, or a combination thereof.

7. The method of claim 5 where the buffer comprises organic or inorganic salts that maintain a pH of 4.0 to 8.0.

8. The method of claim 1 where the combining together the sample comprising red blood cells and the aqueous dextran solution comprises;
adding the sample comprising red blood cells to a mixing vessel, the mixing vessel having two or more valve ports positions for introducing and extracting materials; and
adding the aqueous dextran into the mixing vessel from a receptacle, the receptacle being in fluid communication with the mixing vessel through one of the valve ports.

9. The method of claim 8 further comprising the steps of incubating the sample to aggregate and sediment of the red blood cells and optionally recovering total nucleated cells (TNC) from the sample.

10. The method of claim 8 where incubating the sample occurs in the mixing vessel.

11. The method of claim 10 where recovering the TNC comprises concentration of a liquid phase extracted from the mixing vessel, said liquid phase comprising plasma and dextran using centrifugation, membrane filtration, or a combination thereof.

12. The method of claim 1, wherein the sample comprises whole blood.

13. The method of claim 1, wherein the sample comprises peripheral blood, cord blood or bone marrow.

14. The method of claim 1, wherein the aqueous dextran solution is 1 to 5 wt/v % of dextran.

15. The method of claim 1, wherein the dose of gamma radiation is between 25 and 45 kGy.

16. The method of claim 1, wherein the mineral salt of the ascorbic acid is selected from the group consisting of: sodium ascorbate, calcium ascorbate, potassium ascorbate, magnesium ascorbate, zinc ascorbate and a combination thereof.

17. The method of claim 1, wherein the ascorbic acid or its mineral salt is present in an amount of from 4 to 15 wt %.

18. The method of claim 7, wherein the buffer is phosphate buffered saline (PBS).

19. The method of claim 1, wherein the initial average molecular weight of dextran is between 1000 kD and 2000 kD.

20. The method of claim 1, wherein the initial average molecular weight of dextran is between 1000 kD and 1500 kD.
